(19) United States
(12) Patent Application Publication
(10) Pub. No.: US 2007/0003623 A1
(43) Pub. Date: Jan. 4, 2007

(54) Formulations of conjugated estrogens and bazedoxifene

(75) Inventors: Ramarao S. Chatlapalli, Hopewell Junction, NY (US); Arwinder S. Nagi, Thiells, NY (US); Lawrence Van Pelt, Garnerville, NY (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/478,400

(22) Filed: Jun. 28, 2006

Related U.S. Application Data
(60) Provisional application No. 60/694,889, filed on Jun. 29, 2005.

(51) Int. Cl. A61K 9/24; A61K 31/56

(52) U.S. Cl. 424/472; 514/171

(57) ABSTRACT

The present invention relates to solid dosage formulations containing conjugated estrogens and bazedoxifene, or a salt thereof. In some embodiments, the compositions include a core comprising conjugated estrogens, and at least one coating that comprises bazedoxifene, or a salt thereof.

### CROSS REFERENCE TO RELATED APPLICATIONS

This application claims priority benefit of U.S. Provisional application Ser. No. 60/694,889, filed Jun. 29, 2005, the entire content of which is incorporated by reference herein.

### FIELD OF THE INVENTION

The present invention relates to solid dosage formulations containing conjugated estrogens and bazedoxifene, or a salt thereof. In some embodiments, the compositions include a core containing conjugated estrogens, and a coating that includes bazedoxifene, or a salt thereof.

### BACKGROUND OF THE INVENTION

The use of hormone replacement therapy for bone loss prevention in post-menopausal women is well precedented. The normal protocol calls for estrogen supplementation using such formulations containing estrone, estriol, ethynyl estradiol or conjugated estrogens isolated from natural sources (i.e. Premarin® conjugated estrogens from Wyeth-Ayerst). In some patients, therapy may be contraindicated due to the proliferative effects of unopposed estrogens (estrogens not given in combination with progestins) have on uterine tissue. This proliferation is associated with increased risk for endometriosis and/or endometrial cancer. The effects of unopposed estrogens on breast tissue is less clear, but is of some concern. The need for estrogens which can maintain the bone sparing effect while minimizing the proliferative effects in the uterus and breast is evident.

The use of indoles as estrogen antagonists has been reported by Von Angerer, Chemical Abstracts, Vol. 99, No. 7 (1983), Abstract No. 53886u. Also, see, J. Med. Chem. 1990, 33, 2635-2640; J. Med. Chem. 1987, 30, 131-136. Also see Ger. Offen., DE 3821148 A1 891228 and WO 96/03375. The majority of the compounds reported in these publications fall into a class of compounds best characterized as being “pure antiestrogens”. Additional reports of indole antiestrogens include: WO A 95 17383 (Kar Bio AB), WO A 93 10741, and WO 93/23374 (Otsuka Pharmaceuticals, Japan).

U.S. Pat. No. 5,998,402 describes 2-phenylindoles that are estrogen agonists/antagonists useful for the treatment of diseases associated with estrogen deficiency. The compounds show strong binding to the estrogen receptor. In vitro assays, including an Ishikawa alkaline phosphatase assay and an ERE transfection assay, show these compounds are antiestrogens with little to no intrinsic estrogenicity and they have proven capable of completely antagonizing the effects of 17β-estradiol while showing little or no uterine stimulation in a rat uterine assay when dosed alone. Additionally, some of the compounds are capable of inhibiting bone loss in an ovariectomized rat while showing little or no uterine stimulation. These compounds also decrease the weight gain normally seen in the ovariectomized animals as well as reduce total cholesterol levels. One preferred such compound is bazedoxifene, which is 1-[4-(azepan-1-yl-ethoxy)-benzyl]-2-(4-hydroxy-phenyl)-3-methyl-1H-indole 5-ol.

Bazedoxifene is a tissue selective estrogen for treatment and prevention of postmenstrual osteoporosis. It has been reported to prevent bone loss and protect the cardiovascular system and reduce or eliminate the negative effects on the uterus and breast (potential risk of uterine and breast cancers).

U.S. Pat. Nos. 5,998,402 and 6,479,535 report the preparation of bazedoxifene acetate. The synthetic preparation of bazedoxifene acetate has also appeared in the general literature. See, for example, Miller et al., J. Med. Chem., 2001, 44, 1654-1657. Further description of the drug's biological activity has appeared in the general literature as well (e.g. Miller, et al. Drugs of the Future, 2002, 27(2), 117-121). Formulations of bazedoxifene acetate are also reported in U.S. Pat. App. Pub. No. 2002/0031548 A1.

Bazedoxifene acetate polymorphs are described in U.S. Provisional App. Ser. Nos. 60/560,582 filed Apr. 7, 2004, and 60/560,584 filed Apr. 7, 2004. Bazedoxifene acetate dispersion formulations are described in U.S. Provisional App. Ser. No. 60/560,452, filed Apr. 8, 2004. Bazedoxifene ascorbate salt is described in U.S. Provisional App. Ser. No. 60/560,454, filed Apr. 8, 2004.

A need exists for effective drug treatment for post menopausal women to relieve vasomotor symptoms and protect bones without stimulating the endometrium (which may lead to endometrial hyperplasia) or the breast. The present invention is directed to these, as well as other, important ends.

### SUMMARY OF THE INVENTION

In some embodiments, the present invention provides pharmaceutical compositions including a core and at least one coating; wherein the core comprises conjugated estrogens; and the coating comprises bazedoxifene, or a pharmaceutically acceptable salt thereof. In some embodiments, the conjugated estrogens include or consist of Premarin®.

In some embodiments, the pharmaceutical composition is a tablet. In some such embodiments, the pharmaceutical compositions include a core and at least one coating; wherein the core includes conjugated estrogens; and the coating includes bazedoxifene, or a pharmaceutically acceptable salt thereof.

In some embodiments, the coating comprises:

a) a filler component comprising from about 5% to about 30% by weight of the pharmaceutical formulation;

b) a binder component comprising from about 1% to about 10% by weight of the pharmaceutical formulation;

c) a wetting agent component comprising from about 0.01% to about 2% by weight of the pharmaceutical formulation;

d) an optional antioxidant component comprising from 0% to about 2% by weight of the pharmaceutical formulation;

e) bazedoxifene acetate, comprising from about 0.1% to about 20% by weight of the pharmaceutical formulation; and

f) an optional chelating component comprising from 0% to about 0.1% by weight of the pharmaceutical formulation.

In some embodiments, the conjugated estrogens is present in an amount of from about 0.10 to about 1.0 mg; or from about 0.3 to about 0.8 mg; or from about 0.4 to about 0.5 mg; or from about 0.5 to about 0.7 mg.

In some embodiments, the bazedoxifene is present in an amount of from about 1 to about 50 mg, based on the weight of the bazedoxifene free base; or from about 5 to about 25 mg, based on the weight of the bazedoxifene free base; or from about 5 to about 15 mg, based on the weight of the bazedoxifene free base; or from about 15 to about 25 mg, based on the weight of the bazedoxifene free base; or from about 35 to about 45 mg, based on the weight of the bazedoxifene free base.

In some embodiments, the conjugated estrogens is present in an amount of from about 0.10 to about 1.0 mg; and the bazedoxifene is present in an amount of from about 5 to about 50 mg, based on the weight of the bazedoxifene free base.

In some embodiments, the conjugated estrogens is Premarin®, and the bazedoxifene is bazedoxifene acetate.

In some embodiments, the filler component of the coating comprises sucrose; the binder component of the coating comprises hydroxypropylmethylcellulose; the wetting agent component of the coating comprises sucrose palmitate; the optional antioxidant component of the coating, when present, comprises ascorbic acid, or a salt thereof; and the optional chelating component, when present, comprises EDTA.

In some embodiments, the pharmaceutical compositions further include color coating. In some embodiments, the color coating includes:

a) an optional filler component comprising from about 0.01% to about 8% by weight of the pharmaceutical formulation;

b) an optional binder component comprising from about 0.01% to about 2% by weight of the pharmaceutical formulation; and

c) a coloring agent component comprising from about 0.01% to about 6% by weight of the pharmaceutical formulation. In some such embodiments, the optional filler component comprises sucrose. In some further such embodiments, the optional binder component comprises hydroxypropylmethylcellulose. In some further such embodiments, the coloring agent component comprises titanium dioxide.

In some embodiments, the pharmaceutical compositions further include a clear coating. In some such embodiments, the clear coating forms from about 0.01% to about 2% by weight of the pharmaceutical formulation.

In some embodiments, the optional antioxidant component, the optional chelating component, or both the optional antioxidant component and the optional chelating component, additionally being optionally present in the coating as described above, also can each independently and optionally be present in one or more of the color coating and the clear coating.

The invention further provides processes for the preparation of pharmaceutical compositions of the invention, and products of the processes.

### DETAILED DESCRIPTION

In some embodiments, the present invention provides pharmaceutical compositions including conjugated estrogens and bazedoxifene, or a salt thereof. In some embodiments, the conjugated estrogens include or consist of Premarin®.

The pharmaceutical compositions of the invention include capsules and tablet in capsule (TIC) formulations. In some embodiments, the pharmaceutical compositions of the invention are tablets.

In some embodiments, the pharmaceutical compositions of the invention include a core and at least one coating; wherein the core includes conjugated estrogens, preferably Premarin®, and the coating includes bazedoxifene, or a pharmaceutically acceptable salt thereof, preferably bazedoxifene acetate.

In some embodiments, the coating comprises:

a) a filler component comprising from about 5% to about 30% by weight of the pharmaceutical formulation;

b) a binder component comprising from about 1% to about 10% by weight of the pharmaceutical formulation;

c) a wetting agent component comprising from about 0.01% to about 2% by weight of the pharmaceutical formulation;

d) an optional antioxidant component comprising from 0% to about 2% by weight of the pharmaceutical formulation;

e) bazedoxifene acetate, comprising from about 0.1% to about 20% by weight of the pharmaceutical formulation; and

f) an optional chelating component comprising from 0% to about 0.1% by weight of the pharmaceutical formulation.

In some further embodiments, the coating comprises:

a) a filler component comprising from about 6% to about 12% by weight of the pharmaceutical formulation;

b) a binder component comprising from about 1% to about 6% by weight of the pharmaceutical formulation;

c) a wetting agent component comprising from about 0.1% to about 3% by weight of the pharmaceutical formulation;

d) an optional antioxidant component comprising from 0% to about 0.5% by weight of the pharmaceutical formulation;

e) bazedoxifene acetate, comprising from about 2% to about 6% by weight of the pharmaceutical formulation; and

f) an optional chelating component comprising from 0% to about 0.1% by weight of the pharmaceutical formulation.

In some embodiments, the coating comprises:

a) a filler component comprising from about 12% to about 18% by weight of the pharmaceutical formulation;

b) a binder component comprising from about 4% to about 8% by weight of the pharmaceutical formulation;

c) a wetting agent component comprising from about 0.2% to about 0.5% by weight of the pharmaceutical formulation;

d) an optional antioxidant component comprising from 0% to about 0.8% by weight of the pharmaceutical formulation;

e) bazedoxifene acetate, comprising from about 4% to about 9% by weight of the pharmaceutical formulation; and

f) an optional chelating component comprising from 0% to about 0.1% by weight of the pharmaceutical formulation.

In some further embodiments, the coating comprises:

a) a filler component comprising from about 20% to about 30% by weight of the pharmaceutical formulation;

b) a binder component comprising from about 6% to about 10% by weight of the pharmaceutical formulation;

c) a wetting agent component comprising from about 0.4% to about 0.8% by weight of the pharmaceutical formulation;

d) an optional antioxidant component comprising from 0% to about 1.2% by weight of the pharmaceutical formulation;

e) bazedoxifene acetate, comprising from about 7% to about 14% by weight of the pharmaceutical formulation; and

f) an optional chelating component comprising from 0% to about 0.1% by weight of the pharmaceutical formulation.

In some embodiments, the core of the composition includes conjugated estrogens and one or more fillers and/or binding agents. In some embodiments, the conjugated estrogens include or consist of Premarin®. Generally, the core includes conjugated estrogens in an amount of from about 0.10 to about 1.0 mg, and forms from about 45% to about 80% by weight of the pharmaceutical formulation. In some embodiments, the core includes conjugated estrogens in an amount of from about 0.3 to about 0.8 mg. In some embodiments, the core includes conjugated estrogens in an amount of from about 0.4 to about 0.5 mg; for example about 0.45 mg, or from about 0.5 to about 0.7 mg, for example about 0.625 mg. In some embodiments, the core is a filled tablet containing conjugated estrogens, preferably Premarin®.

In some embodiments, the pharmaceutical compositions of the invention include at least one coating that contains bazedoxifene, or a pharmaceutically acceptable salt thereof. In some embodiments, the bazedoxifene, or pharmaceutically acceptable salt thereof, is present in an amount of from about 1 to about 50 mg, based on the weight of the bazedoxifene free base. As used herein, the term “based on the weight of the bazedoxifene free base” is intended to mean that amount of bazedoxifene or salt thereof that provides the same number of molecules of bazedoxifene as the indicated mass of bazedoxifene free base. Thus, for example, the phrase “10 mg of bazedoxifene acetate, based on the weight of the bazedoxifene free base” would indicate a mass of bazedoxifene acetate sufficient to provide a number of molecules of bazedoxifene acetate that is the same as the number of bazedoxifene molecules present in 10 mg of the free base form of bazedoxifene. In some embodiments, the bazedoxifene, or pharmaceutically acceptable salt thereof, is present in an amount of from about 5 to about 25 mg, or from about 5 to about 15 mg, or from about 15 to about 25 mg, or from about 35 to about 45 mg, based on the weight of the bazedoxifene free base.

In some embodiments, the bazedoxifene is present in the pharmaceutical formulation as the acetate salt. In some such embodiments, the bazedoxifene acetate present in the coating forms from about 0.1% to about 20% by weight of the pharmaceutical formulation; or from about 2% to about 6% by weight of the pharmaceutical formulation; or from about 4% to about 9% by weight of the pharmaceutical formulation; or from about 7% to about 14% by weight of the pharmaceutical formulation.

In addition to the bazedoxifene, or a pharmaceutically acceptable salt thereof, the coating can contain one or more of fillers, diluents, binders, wetting agents, and/or antioxidants.

Generally, the filler component of the coating forms from about 5% to about 30% by weight of the pharmaceutical formulation; or from about 6% to about 12% by weight of the pharmaceutical formulation; or from about 12% to about 18% by weight of the pharmaceutical formulation; or from about 20% to about 30% by weight of the pharmaceutical formulation. The filler component can include one or more fillers known to be useful in the art, for example one or more of sugars, for example sucrose, mannitol, lactose, and the like, and/or other fillers such as powdered cellulose, microcrystalline cellulose, malodextrin, sorbitol, starch, xylitol, carboxymethyl cellulose, carboxyethyl cellulose, hydroxyethyl celluloses, microcrystalline celluloses, starches, anhydrous dicalcium phosphate, sodium starch glycolates, and metal aluminosilicates. In some embodiments, the filler component of the coating includes one or more sugars. As used herein, the term “sugar” refers to any type of simple carbohydrate, such as a mono or disaccharide, either naturally obtained, refined from a natural source, or artificially produced, and includes, without limitation, sucrose, dextrose, maltose, glucose, fructose, galactose, mannose, lactose, trehalose, lactulose, levulose, raffinose, ribose, and xylose. The term “sugar,” as used herein, also includes various “sugar substitutes” widely known to those of ordinary skill in the art of preparing solid dosage forms, such as the polyhydric alcohols (sometimes referred to as “sugar alcohols” or hydrogenated saccharides), for example sorbitol, mannitol, xylitol, and erythritol, and the sugar derivatives of polyhydric alcohols, such as maltitol, lactitol, isomalt, and polyalditol. Accordingly, the recitation of the term “sugar” generically should be interpreted to include such specific compounds, as well as others not expressly recited. In certain embodiments, the sugar is a mono- or disaccharide, for example, sucrose, dextrose, maltose, glucose, fructose, galactose, mannose, or lactose. In some preferred embodiments, the filler component of the coating includes or consists of sucrose.

Generally, the binder component of the coating forms from about 1% to about 10% by weight of the pharmaceutical formulation; or from about 1% to about 6% by weight of the pharmaceutical formulation; or from about 4% to about 8% by weight of the pharmaceutical formulation; or from about 6% to about 10% by weight of the pharmaceutical formulation. The binder component can include one or more binders known to be useful in the art, for example one or more of hydroxypropylmethylcellulose, carboxymethyl cellulose, carboxyethyl cellulose, hydroxyethyl celluloses, microcrystalline celluloses, starches and polyvinyl pyrrolidine (PVP). In some preferred embodiments, the filler component includes or consists of hydroxypropyl methylcellulose.

It has been found in accordance with the present invention that sucrose, although not a typical film forming agent like hydroxypropyl methylcellulose, is particularly advantageous when used with a lower viscosity grade of hydroxypropyl methylcellulose, for example 3 cps. While not wishing to be bound by any particular theory, the sucrose is believed to add body to the coating, and to act as a soluble-filler in an active overcoat process. Generally, it is beneficial to employ a ratio of hydroxypropyl methylcellulose to sucrose of from about 1:2 to about 1:5, or from about 1:2 to about 1:4; or from about 1:2.5 to about 1:3.5; or about 1:3. Such a ratio is believed to provide the most acceptable viscosity and sprayability characteristics of the filler suspension at 20% w/w solids level for a continuous coating process.

Generally, the wetting agent component of the coating is selected to increase wettability of the components of the filler coating, and in particular, bazedoxifene, and to therefore aid in dispersing the bazedoxifene. Preferably, the wetting agent possesses low foaming characteristics, and preferably has antimicrobial activity. Generally, the wetting agent component of the coating forms from about 0.01% to about 2% by weight of the pharmaceutical formulation; or from about 0.1% to about 3% by weight of the pharmaceutical formulation; or from about 0.2% to about 0.5% by weight of the pharmaceutical formulation; or from about 0.4% to about 0.8% by weight of the pharmaceutical formulation. The wetting agent component can include one or more wetting agents known to be useful in the art, for example one or more of sucrose fatty acid esters, such as sucrose palmitate, and Poloxamer 188, metal alkyl sulfates, sodium lauryl sulfate, polyoxyethylene sorbitan fatty acid esters, polyethylene glycols, polyoxyethylene castor oil derivatives, docusate sodium, quaternary ammonium amine compounds, sugar esters of fatty acids and glycerides of fatty acids. In some preferred embodiments, the wetting agent component includes or consists of sucrose palmitate.

Generally, the optional antioxidant component of the coating forms up to about 15% by weight, e.g., from 0% to about 15% by weight of the pharmaceutical formulation, from about 0.01% to about 5% by weight of the pharmaceutical formulation; from about 0.01% to about 2% by weight of the pharmaceutical formulation; or from about 0.1% to about 0.5% by weight of the pharmaceutical formulation; or from about 0.3% to about 0.8% by weight of the pharmaceutical formulation; or from about 0.6% to about 1.2% by weight of the pharmaceutical formulation. The antioxidant component, when present, can include one or more antioxidants known to be useful in the art, for example one or more of ascorbic acid or a salt thereof such as sodium ascorbate, ascorbyl palmitate, nicotinamide ascorbate, propyl gallate, tocopherol (alpha, beta and gamma), BHA/BHT, citric acid and salts thereof, for example sodium citrate. In some preferred embodiments, the antioxidant component includes or consists of ascorbic acid.

Further examples of suitable fillers, binders, wetting agents and antioxidants can be found in, for example, Remington's Pharmaceutical Sciences, 17th ed., Mack Publishing Company, Easton, Pa., 1985, which is incorporated herein by reference in its entirety.

Generally, the optional chelating component forms up to about 0.1% by weight of the pharmaceutical formulation. In some embodiments, the chelating component is present in an amount of from about 0.01% to about 0.10% by weight of the coating. The chelating component can include one or more chelating agents as are known in the art for use in pharmaceutical formulations. One preferred chelating agent is ethylenediaminetetraacetic acid (EDTA). Other suitable chelating agents can be found in, for example, Remington's Pharmaceutical Sciences, supra.

In some embodiments, the optional antioxidant component, the optional chelating component, or both the optional antioxidant component and the optional chelating component, additionally being optionally present in the coating as described above, also can each independently and optionally be present in one or more of the color coating and the clear coating, as described below. In some embodiments, the antioxidant component and the chelating component are both present together in one or more of the coatings. Thus, in some such embodiments, the antioxidant component and the chelating component are both present in the coating containing the active agent, e.g., bazedoxifene; or in the color coating; or in the clear coating; or in any two of the coatings, or in all three of the coatings.

In some embodiments, the pharmaceutical composition further includes a color coating. Generally, the color coating is formed over the first coating described above, and contains at least one coloring agent. In some embodiments, the color coating includes:

a) an optional filler component including from about 0.01% to about 8% by weight of the pharmaceutical formulation;

b) an optional binder component including from about 0.01% to about 2% by weight of the pharmaceutical formulation; and

c) a coloring agent component including from about 0.01% to about 6% by weight of the pharmaceutical formulation.

The color coating can also optionally include the aforementioned antioxidant component, or the aforementioned chelating component, or both, as described above.

Generally, the optional filler component of the color coating can include one or more fillers as described above for the first coating. In some embodiments, the filler component of the color coating, when present, includes or consists of sucrose.

Generally, the optional binder component of the color coating can include one or more binders as described above for the first coating. In some embodiments, the binder component of the color coating, when present, includes or consists of hydroxypropylmethylcellulose.

The coloring agent component can include one or more of any of the variety of coloring agents known to be useful in the pharmaceutical arts. In some embodiments, the coloring agent component includes or consists of titanium dioxide. Further preferred coloring agents include, for example, Opadry® agents, for example Opadry® White YS-1-18202 A

In some embodiments, the pharmaceutical compositions of the invention further include a clear coating. In some embodiments, the clear coating includes from about 0.01% to about 2% by weight of the pharmaceutical formulation. Generally, the clear coating is formed over the color coating, when present, as described above, or, alternatively, directly on the first coating as described above. Any of the numerous clear coatings known in the art are suitable for use in the pharmaceutical compositions of the invention, for example Opadry® coatings, for example Opadry® Clear YS-2-19114 A.

The clear coating can also optionally include the aforementioned antioxidant component, or the aforementioned chelating component, or both, as described above.

In some embodiments, the invention provides processes for preparing pharmaceutical compositions of the invention. In some embodiments, the processes are used to prepare pharmaceutical compositions of the invention that include:

a core including conjugated estrogens; and

a first coating including bazedoxifene or a pharmaceutically acceptable salt thereof. In some embodiments, the processes include:

i) providing a core including conjugated estrogens; and

ii) coating the core with a coating composition including bazedoxifene or a pharmaceutically acceptable salt thereof to form a coated core.

In some embodiments, the processes further include the step of:

iii) coating the coated core with a color coating composition to form a color coated composition.

In some embodiments, the processes further include the step of:

iv) coating the color coating composition with a clear coating composition to form a clear coat thereon.

Traditionally, sugar coatings are applied to tablet formulations using an intermittent process. In the intermittent active sugar coating process, discrete amounts of the active sugar coat suspension are applied to the surface of a dosage form, for example a tablet, followed by a distribution phase and drying phase, which is repeated several hundred times until the desired weight gain is achieved. Several products currently sold are manufactured using this technique. This process, however, has some limitations. Examples of such limitations are the limited drug loading capacity to keep a reasonable tablet size and process times, and the limitation of the available excipients that can be used to modify the release rate and inherent variability of the process.

While such intermittent processes are amenable to the present formulations, it has been found in accordance with the present invention that sugar coatings can be applied to tablets or other coatable dosage forms by a continuous process. In the continuous sugar coating process, active suspension is applied in a “continuous manner” which is similar to traditional film coating process from the process stand-point. It is known that sugar, as such in a solution, can not be sprayed continuously on to tablets because of its inherent physico-chemical properties, such as solubility, viscosity, and its crystallization kinetics when dried by spray application on to tablets. However, use of the formulations described herein, with appropriate control of process variables, produces a product of acceptable quality and stability, and addresses the limitations of the intermittent sugar coating process described above.

Accordingly, in some embodiments, the coating of the core with the coating composition including bazedoxifene or a pharmaceutically acceptable salt thereof is performed by a continuous process.

In some embodiments, the coating composition of step ii) includes:

a) a filler component;

b) a binder component;

c) a wetting agent component;

d) an optional antioxidant component;

e) bazedoxifene acetate; and

f) an optional chelating component.

wherein the filler component, the binder component, the wetting agent component, the optional antioxidant component, and the optional chelating component are as described above. In some preferred embodiments, the filler component of the coating comprises sucrose; the binder component of the coating comprises hydroxypropylmethylcellulose; the wetting agent component of the coating comprises sucrose palmitate; the optional antioxidant component of the coating, when present, comprises ascorbic acid, or a salt thereof; and the optional chelating component, when present, comprises EDTA.

In some embodiments, the color coating composition includes:

an optional filler component;

an optional binder component; and

a coloring agent component;

wherein the optional filler component, the optional binder component, and the coloring agent component are as described above. The color coating can also optionally include the aforementioned antioxidant component, or the aforementioned chelating component, or both, as described above.

In some embodiments, the optional filler component of the color coating composition includes sucrose; the optional binder component of the color coating composition includes hydroxypropylmethylcellulose; and the coloring agent component of the color coating composition includes titanium dioxide. In some embodiments, the optional antioxidant component of the color coating, when present, comprises ascorbic acid, or a salt thereof; and the optional chelating component of the color coating, when present, comprises EDTA.

In some embodiments, the filler component of the coating composition of step ii) forms from about 5% to about 30% by weight of the pharmaceutical formulation; the binder component of the coating composition of step ii) forms from about 1% to about 10% by weight of the pharmaceutical formulation; the wetting agent component of the coating composition of step ii) forms from about 0.01% to about 2% by weight of the pharmaceutical formulation; the optional antioxidant component of the coating composition of step ii) forms from about 0% to about 2% by weight of the pharmaceutical formulation; the bazedoxifene acetate includes forms from about 0.1% to about 20% by weight of the pharmaceutical formulation; and the optional chelating component of the coating forms from 0% to about 0.1% by weight of the pharmaceutical formulation. In some embodiments, the conjugated estrogens is present in an amount of from about 0.10 to about 1.0 mg. In some further embodiments, the bazedoxifene is present in an amount of from about 1 to about 50 mg, based on the weight of the bazedoxifene free base. In some further embodiments, the conjugated estrogens is present in an amount of from about 0.10 to about 1.0 mg; and the bazedoxifene is present in an amount of from about 5 to about 50 mg, based on the weight of the bazedoxifene free base.

In some embodiments of each of the pharmaceutical compositions and processes of the invention, the conjugated estrogens include or consist of Premarin®.

In some embodiments of each of the pharmaceutical compositions and processes of the invention, the conjugated estrogens is present in the composition in an amount of from about 0.10 to about 1.0 mg; or from about 0.3 to about 0.8 mg; or from about 0.4 to about 0.5 mg; or from about 0.5 to about 0.7 mg.

In some embodiments of each of the pharmaceutical compositions and processes of the invention, the bazedoxifene is present in an amount of from about 1 to about 50 mg, or from about 5 to about 25 mg, or from about 5 to about 15 mg, or from about 15 to about 25 mg, or from about 35 to about 45 mg, based on the weight of the bazedoxifene free base.

In some embodiments of each of the pharmaceutical compositions and processes of the invention, the conjugated estrogens is present in an amount of from about 0.10 to about 1.0 mg; and the bazedoxifene is present in an amount of from about 5 to about 50 mg, based on the weight of the bazedoxifene free base.

In some embodiments of each of the pharmaceutical compositions and processes of the invention, the conjugated estrogens is present in the composition in an amount of from about 0.4 to about 0.5 mg; or from about 0.5 to about 0.7 mg; and the bazedoxifene is present in an amount of from about 5 to about 15 mg, or from about 15 to about 25 mg, or from about 35 to about 45 mg, based on the weight of the bazedoxifene free base.

The present invention also provides products of the processes described herein.

It will be understood that the weight percentages set forth for each of the core, filler components, a binder components, wetting agent components, optional antioxidant components, optional chelating components, optional filler components, optional binder components, coloring agent components and clear coatings of the compositions disclosed herein are the percentages that each component will comprise of a final pharmaceutical composition, including, if present, the clear and color coatings.

Oral formulations containing the present solid dispersions can comprise a variety of conventionally used oral forms, for example tablets and tablet-in-capsule forms. Generally, tablets and tablet-in-capsule forms are preferred. Capsules or tablets of containing the present solid dispersion can also be combined with mixtures of other active compounds or inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. In some preferred embodiments, the formulations are tablets.

Tablet formulations can be made by conventional compression, wet granulation, or dry granulation methods and utilize pharmaceutically acceptable diluents (fillers), binding agents, lubricants, disintegrants, suspending or stabilizing agents, including those described above, as well as, without limitation, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations used herein may utilize standard delay or time release formulations or spansules. Suppository formulations may be made from traditional materials, including cocoa butter, with or without the addition of waxes to alter the suppositories melting point, and glycerin. Water soluble suppository bases, such as polyethylene glycols of various molecular weights, may also be used.

Film coatings useful with the present formulations are known in the art and generally consist of a polymer (usually a cellulosic type of polymer), a colorant and a plasticizer. Additional ingredients such as wetting agents, sugars, flavors, oils and lubricants can be included in film coating formulations to impart certain characteristics to the film coat. The compositions and formulations herein may also be combined and processed as a solid, then placed in a capsule form, such as a gelatin capsule.

As will be appreciated, some components of the formulations of the invention can possess multiple functions. For example, a given component can act as both a binder and a filler. In some such cases, the function of a given component can be considered singular, even though its properties may allow multiple functionality.

Additional numerous various excipients, dosage forms, dispersing agents and the like that are suitable for use in connection with the solid dispersions of the invention are known in the art and described in, for example, Remington's Pharmaceutical Sciences, 17th ed., Mack Publishing Company, Easton, Pa., 1985, which is incorporated herein by reference in its entirety.

As discussed above, it has been found in accordance with the present invention that sugar coatings can be applied to tablets or other coatable dosage forms by a continuous process. Accordingly, in some embodiments, the present invention provides processes for preparing a pharmaceutical composition, the composition comprising:

a core comprising a therapeutic agent; and

a coating optionally comprising a second therapeutic agent and at least one sugar;

the process comprising:

i) providing a core comprising the first therapeutic agent; and

ii) coating the core with a coating composition comprising:
a) a filler component that comprises at least one sugar;
b) a binder component;
c) a wetting agent component;
d) an optional antioxidant component;
e) optionally, a second therapeutic agent; and
f) an optional chelating component;

wherein the coating composition of step ii) is applied by a continuous sugar coating technique.

In some such embodiments:

the filler component comprises one or more of sucrose, mannitol, lactose, powdered cellulose, microcrystalline cellulose, malodextrin, sorbitol, starch, xylitol, carboxymethyl cellulose, carboxyethyl cellulose, hydroxyethyl celluloses, microcrystalline celluloses, starches, anhydrous dicalcium phosphate, sodium starch glycolates, and metal aluminosilicates;

the binder component comprises one or more of hydroxypropylmethylcellulose, carboxymethyl cellulose, carboxyethyl cellulose, hydroxyethyl celluloses, microcrystalline celluloses, starches;

the wetting agent component comprises one or more of sucrose palmitate, Poloxamer 188, metal alkyl sulfates, sodium lauryl sulfate, polyoxyethylene sorbitan fatty acid esters, polyethylene glycols, polyoxyethylene castor oil derivatives, docusate sodium, quaternary ammonium amine compounds, sugar esters of fatty acids and glycerides of fatty acids; and

the optional antioxidant component, when present, comprises one or more of ascorbic acid citric acid, sodium ascorbate, ascorbyl palmitate, nicotinamide ascorbate, propyl gallate, alpha tocopherol, beta tocopherol, gamma tocopherol, and BHA/BHT; and

the optional chelating component, when present, comprises EDTA.

In some further embodiments, the filler component of the coating comprises sucrose; the binder component of the coating comprises hydroxypropylmethylcellulose; the wetting agent component of the coating comprises sucrose palmitate; the optional antioxidant component of the coating comprises ascorbic acid, or a salt thereof; and the optional chelatin component, when present, comprises EDTA.

In some further embodiments, the processes further comprising the step of iii) coating the coated core with a color coating composition to form a color coated composition; and in some further embodiments, the processes further comprising the step of iv) coating the color coated composition with a clear coating composition to form a clear coat thereon.

The first therapeutic agent can be any of a wide variety of therapeutic agents. As used herein, the term “therapeutic agent” also refers to a substance which is capable of exerting a therapeutic biological effect in vivo. The therapeutic agents may be neutral or positively or negatively charged. Examples of suitable pharmaceutical agents include, inter alia, diagnostic agents, pharmaceuticals, drugs, synthetic organic molecules, proteins, peptides, vitamins, and steroids. For example, the composition may include one or more hormonal steroids, such as medroxyprogesterone acetate, levonorgestrel, gestodene, medrogestone, estradiol, estriol, ethinylestradiol, mestranol, estrone, dienestrol, hexestrol, diethylstilbestrol, progesterone, desogestrel, norgestimate, hydroxyprogesterone, norethindrone, norethindone acetate, norgestrel, megestrol acetate, methyltestosterone, ethylestrenol, methandienone, oxandrolone, trimegestone, dionogest, and the like. Additionally, tissue selective progesterones and/or progesterone antagonists, which may or may not have the typical steroidal functionality, may be present in the composition. These include, but are not limited to: RU-486 (mifepristone), ZK 98 299 (onapristone), ZK-137316 (Schering AG, Berlin), ZK-230211 (Schering AG, Berlin), and HRP-2000 (17-acetoxy-[11β-(4-N,N-dimethylaminophenyl)]-19-norpregna-4,9-diene-3,20-dione). Where desired, estrogenic steroids and progestogenic steroids may be used in combination.

The processes of the present invention are particularly suitable for use in coating core materials, for example tablets, to produce a coated solid dosage form. The term “core material” refers to any tablet, caplet, particle, micronized particle, particulate, pellet, pill, core, granule, granulate, small mass, seed, specks, spheres, crystals, beads, agglomerates, mixtures thereof and the like that are sufficiently stable physically and chemically to be effectively coated in a continuous sugar coating process.

In a preferred embodiment, the core material is present in the form of a tablet. As used herein, the term “tablet” refers to a solid pharmaceutical dosage form containing a therapeutic agent with or without suitable diluents and prepared by either compression or molding methods, such as are well known to those of ordinary skill in the art. Suitable methods of forming tablets are described, for example, in Edward M Rudnick, et al., “Oral Solid Dosage Forms,” in Remington: The Science and Practice of Pharmacy, 20^{th} Ed., Chap. 45, Alfonso R. Gennaro, ed., Philadelphia College of Pharmacy and Science, Philadelphia, Pa. (2000), herein incorporated by reference in its entirety. In some embodiments, the core material is a tablet formed by compression methods.

Most frequently, the core material will comprise at least one therapeutic agent, as defined previously, and at least one pharmaceutically acceptable excipient. The term “pharmaceutically acceptable,” as used herein, refers to materials that are generally not toxic or injurious to a patient when used in the compositions of the present invention, including when the compositions are administered by the oral route. The term “patient,” as used herein, refers to animals, including mammals, preferably humans. “Excipients,” as that term is used herein, refers to ingredients that provide bulk, impart satisfactory processing and compression characteristics, help control the dissolution rate, and/or otherwise give additional desirable physical characteristics to the core material. Included within this term, for example, are diluents, binders, lubricants and disintegrants well known to those of ordinary skill in the art, as described, for example, in the Handbook of Pharmaceutical Excipients, American Pharmaceutical Association, Washington, D.C. and The Pharmaceutical Society of Great Britain, London, England (1986), herein incorporated by reference in its entirety.

A wide variety of therapeutic agents may be utilized in either the core material i.e., as the first therapeutic agent), or in the coating (i.e., as the second therapeutic agent). Specific examples of therapeutic agents include, but are not limited to: acetazolamide, acetohexamide, acrivastine, alatrofloxacin, albuterol, alclofenac, aloxiprin, alprostadil, amodiaquine, amphotericin, amylobarbital, aspirin, atorvastatin, atovaquone, baclofen, barbital, benazepril, bezafibrate, bromfenac, bumetanide, butobarbital, candesartan, capsaicin, captopril, cefazolin, celecoxib, cephadrine, cephalexin, cerivastatin, cetrizine, chlorambucil, chlorothiazide, chlorpropamide, chlorthalidone, cinoxacin, ciprofloxacin, clinofibrate, cloxacillin, cromoglicate, cromolyn, dantrolene, dichlorophen, diclofenac, dicloxacillin, dicumarol, diflunisal, dimenhydrinate, divalproex, docusate, dronabinol, enoximone, enalapril, enoxacin, enrofloxacin, epalrestat, eposartan, essential fatty acids, estramustine, ethacrynic acid, ethotoin, etodolac, etoposide, fenbufen, fenoprofen, fexofenadine, fluconazole, flurbiprofen, fluvastatin, fosinopril, fosphenyloin, fumagillin, furosemide, gabapentin, gemfibrozil, gliclazide, glipizide, glybenclamide, glyburide, glimepiride, grepafloxacin, ibufenac, ibuprofen, imipenem, indomethacin, irbesartan, isotretinoin, ketoprofen, ketorolac, lamotrigine, levofloxacin, lisinopril, lomefloxacin, losartan, lovastatin, meclofenamic acid, mefenamic acid, mesalamine, methotrexate, metolazone, montelukast, nalidixic acid, naproxen, natamycin, nimesulide, nitrofurantoin, non-essential fatty acids, norfloxacin, nystatin, ofloxacin, oxacillin, oxaprozin, oxyphenbutazone, penicillins, pentobarbital, perfloxacin, phenobarbital, phenyloin, pioglitazone, piroxicam, pramipexol, pranlukast, pravastatin, probenecid, probucol, propofol, propylthiouracil, quinapril, rabeprazole, repaglinide, rifampin, rifapentine, sparfloxacin, sulfabenzamide, sulfacetamide, sulfadiazine, sulfadoxine, sulfamerazine, sulfamethoxazole, sulfafurazole, sulfapyridine, sulfasalazine, sulindac, sulphasalazine, sulthiame, telmisartan, teniposide, terbutaline, tetrahydrocannabinol, tirofiban, tolazamide, tolbutamide, tolcapone, tolmetin, tretinoin, troglitazone, trovafloxacin, undecenoic acid, ursodeoxycholic acid, valproic acid, valsartan, vancomycin, verteporfin, vigabatrin, vitamin K-S (II) and zafirlukast. Additional therapeutic agents include abacavir, acebutolol, acrivastine, alatrofloxacin, albuterol, albendazole, alfentanil, alprazolam, alprenolol, amantadine, amiloride, aminoglutethimide, amiodarone, amitriptyline, amlodipine, amodiaquine, amoxapine, amphetamine, amphotericin, amprenavir, amrinone, amsacrine, apomorphine, astemizole, atenolol, atropine, azathioprine, azelastine, azithromycin, baclofen, benethamine, benidipine, benzhexol, benznidazole, benztropine, biperiden, bisacodyl, bisanthrene, bromazepam, bromocriptine, bromperidol, brompheniramine, brotizolam, bupropion, butenafine, butoconazole, cambendazole, camptothecin, carbinoxamine, cephadrine, cephalexin, cetrizine, cinnarizine, chlorambucil, chlorpheniramine, chlorproguanil, chlordiazepoxide, chlorpromazine, chlorprothixene, chloroquine, cimetidine, ciprofloxacin, cisapride, citalopram, clarithromycin, clemastine, clemizole, clenbuterol, clofazimine, clomiphene, clonazepam, clopidogrel, clozapine, clotiazepam, clotrimazole, codeine, cyclizine, cyproheptadine, dacarbazine, darodipine, decoquinate, delavirdine, demeclo-cycline, dexamphetamine, dexchlorpheniramine, dexfenfluramine, diamorphine, diazepam, diethylpropion, dihydrocodeine, dihydroergotamine, diltiazem, dimenhydrinate, diphenhydramine, diphenoxylate, diphenyl-imidazole, diphenylpyraline, dipyridamole, dirithromycin, disopyramide, dolasetron, domperidone, donepezil, doxazosin, doxycycline, droperidol, econazole, efavirenz, ellipticine, enalapril, enoxacin, enrofloxacin, eperisone, ephedrine, ergotamine, erythromycin, ethambutol, ethionamide, ethopropazine, etoperidone, famotidine, felodipine, fenbendazole, fenfluramine, fenoldopam, fentanyl, fexofenadine, flecainide, flucytosine, flunarizine, flunitrazepam, fluopromazine, fluoxetine, fluphenthixol, fluphenthixol decanoate, fluphenazine, fluphenazine decanoate, flurazepam, flurithromycin, frovatriptan, gabapentin, granisetron, grepafloxacin, guanabenz, halofantrine, haloperidol, hyoscyamine, imipenem, indinavir, irinotecan, isoxazole, isradipine, itraconazole, ketoconazole, ketotifen, labetalol, lamivudine, lanosprazole, leflunomide, levofloxacin, lisinopril, lomefloxacin, loperamide, loratadine, lorazepam, lormetazepam, lysuride, mepacrine, maprotiline, mazindol, mebendazole, meclizine, medazepam, mefloquine, melonicam, meptazinol, mercaptopurine, mesalamine, mesoridazine, metformin, methadone, methaqualone, methylphenidate, methylphenobarbital, methysergide, metoclopramide, metoprolol, metronidazole, mianserin, miconazole, midazolam, miglitol, minoxidil, mitomycins, mitoxantrone, modafinil, molindone, montelukast, morphine, moxifloxacin, nadolol, nalbuphine, naratriptan, natamycin, nefazodone, nelfinavir, nevirapine, nicardipine, nicotine, nifedipine, nimodipine, nimorazole, nisoldipine, nitrazepam, nitrofurazone, nizatidine, norfloxacin, nortriptyline, nystatin, ofloxacin, olanzapine, omeprazole, ondansetron, omidazole, oxamniquine, oxantel, oxatomide, oxazepam, oxfendazole, oxiconazole, oxprenolol, oxybutynin, oxyphencyclimine, paroxetine, pentazocine, pentoxifylline, perchlorperazine, perfloxacin, perphenazine, phenbenzamine, pheniramine, phenoxybenzamine, phentermine, physostigmine, pimozide, pindolol, pizotifen, pramipexol, pranlukast, praziquantel, prazosin, procarbazine, prochlorperazine, proguanil, propranolol, pseudoephedrine, pyrantel, pyrimethamine, quetiapine, quinidine, quinine, raloxifene, ranitidine, remifentanil, repaglinide, reserpine, ricobendazole, rifabutin, rifampin, rifapentine, rimantadine, risperidone, ritonavir, rizatriptan, ropinirole, rosiglitazone, roxatidine, roxithromycin, salbutamol, saquinavir, selegiline, sertraline, sibutramine, sildenafil, sparfloxacin, spiramycins, stavudine, sufentanil, sulconazole, sulphasalazine, sulpiride, sumatriptan, tacrine, tamoxifen, tamsulosin, temazepam, terazosin, terbinafine, terbutaline, terconazole, terfenadine, tetramisole, thiabendazole, thioguanine, thioridazine, tiagabine, ticlopidine, timolol, tinidazole, tioconazole, tirofiban, tizanidine, tolterodine, topotecan, toremifene, tramadol, trazodone, triamterene, triazolam, trifluoperazine, trimethoprim, trimipramine, tromethamine, tropicamide, trovafloxacin, vancomycin, venlafaxine, vigabatrin, vinblastine, vincristine, vinorelbine, vitamin K₅, vitamin K₆, vitamin K₇, zafirlukast, zolmitriptan, zolpidem and zopiclone. Of course, any of the foregoing therapeutic agents may be included in the coating composition, as discussed previously, and any of the therapeutic agents discussed with regard to the coating composition alternatively may be included in the core material.

The core material may be designed for delivering therapeutic agents intended to be delivered over a sustained period of time. The following are representative of such therapeutic agents: anti-inflammatory, antipyretic, anti-spasmodics or analgesics such as indomethacin, diclofenac, diclofenac sodium, codeine, ibuprofen, phenylbutazone, oxyphenbutazone, mepirizole, aspirin, ethenzamide, acetaminophen, aminopyrine, phenacetin, butylscopolamine bromide, morphine, etomidoline, pentazocine, fenoprofen calcium, naproxen, selecxip, valdecxip, and tolamadol, anti-rheumatism drugs such as etodolac, anti-tuberculoses drugs such as isoniazide and ethambutol hydrochloride, cardiovascular drugs such as isosorbide dinitrate, nitroglycerin, nifedipine, barnidipine hydrochloride, nicardipine hydrochloride, dipyridamole, amrinone, indenolol hydrochloride, hydralazine hydrochloride, methyldopa, furosemide, spironolactone, guanethidine nitrate, reserpine, amosulalol hydrochloride, lisinopril, metoprolol, pilocarpine, and talcetin, antipsychotic drugs such as chlorpromazine hydrochloride, amitriptyline hydrochloride, nemonapride, haloperidol, moperone hydrochloride, perphenazine, diazepam, lorazepam, chlorodiazepoxide, adinazolam, alprazolam, methylphenidate, myrnasipran, peroxetin, risperidone, and sodium valproate, anti-emetics such as metoclopramide, lamocetron hydrochloride, granisetron hydrochloride, ondansetron hydrochloride, and azacetron hydrochloride, antihistamines such as chlorpheniramine maleate and diphenhydramine hydrochloride, vitamins such as thiamine nitrate, tocopherol acetate, cycothiamine, pyridoxal phosphate, cobarnamide, ascortic acid, and nicotinamide, anti-gout drugs such as allopurinol, colchicine, and probenecide, anti-Parkinson's disease drugs such as levodopa and selegrine, sedatives and hypnotics such as amobarbital, bromuralyl urea, midazolam, and chloral hydrate, antineoplastics such as fluorouracil, carmofur, acralvidine hydrochloride, cyclophosphamide, and thiodepa, anti-allergy drugs such as pseudoephedrine and terfenadine, decongestants such as phenylpropanolamine and ephedorine, diabetes mellitus drugs such as acetohexamide, insulin, tolbutamide, desmopressin, and glipizide, diuretics such as hydrochlorothiazide, polythiazide, and triamterene, bronchodilators such as aminophylline, formoterol fumarate, and theophylline, antitussives such as codeine phosphate, noscapine, dimorfan phosphate, and dextromethorphan, anti-arrhythmics such as quinidine nitrate, digitoxin, propafenone hydrochloride, and procainamide, topical anesthetics such as ethyl aminobenzoate, lidocaine, and dibucaine hydrochloride, anti-convulsants such as phenyloin, ethosuximide, and primidone, synthetic glucocorticoids such as hydrocortisone, prednisolone, triamcinolone, and betamethasone, antiulceratives such as famotidine, ranitidine hydrochloride, cimetidine, sucralfate, sulpiride, teprenone, plaunotol, 5-aminosalicylic acid, sulfasalazine, omeprazole, and lansoprazol, central nervous system drugs such as indeloxazine, idebenone, thiapride hydrochloride, bifemelane hydrocide, and calcium homopantothenate, antihyperlipoproteinemics such as pravastatin sodium, simvastatin, lovastatin, and atorvastatin, antibiotics such as ampicillin hydrochloride, phthalylsulfacetamide, cefotetan, and josamycin, BPH therapeutic agents such as tamsulosin hydrochloride, doxazosin mesylate, and terazosin hydrochloride, drugs affecting uterine motility such as branylcast, zafylcast, albuterol, ambroxol, budesonide, and reproterol, peripheral circulation improvers of prostaglandin I derivatives such as beraprost sodium, anticoagulants, hypotensives, agents for treatment of cardiac insufficiency, agents used to treat the various complications of diabetes, peptic ulcer therapeutic agents, skin ulcer therapeutic agents, agents used to treat hyperlipemia, tocolytics, etc. The therapeutic agent can be used in its free form or as a pharmaceutically acceptable salt. Moreover, one or a combination of two or more therapeutic agents may be present in the core material.

In some embodiments, the therapeutic agent in the core material includes conjugated estrogens. “Conjugated estrogens” (CE) as used herein includes both natural and synthetic conjugated estrogens, such as the compounds described in the United States Pharmacopia (USP 23), as well as other estrogens so considered by those skilled in the art. Further, “conjugated estrogens” refers to esters of such compounds, such as the sulfate esters, salts of such compounds, such as sodium salts, and esters of the salts of such compounds, such as sodium salts of a sulfate ester, as well as other derivatives known in the art. Some specific examples include: 17-alpha and beta-dihydroequilin, equilenin, 17-alpha and beta-dihydroequilenin, estrone, 17-beta-estradiol, and their sodium sulfate esters.

Although CE are typically a mixture of estrogenic components, such as estrone and equilin, the core material may be formulated to either utilize such a mixture, or to include only selected or individual estrogenic components. These CE may be of synthetic or natural origin. Examples of synthetically produced estrogens include, inter alia, sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate, estropipate and ethinyl estradiol. The alkali metal salts of 8,9-dehydroestrone and the alkali metal salts of 8,9-dehydroestrone sulfate ester, as described in U.S. Pat. No. 5,210,081, which is herein incorporated by reference, also may be used. Naturally occurring CE are usually obtained from pregnant mare urine and then are processed and may be stabilized. Examples of such processes are set forth in U.S. Pat. Nos. 2,565,115 and 2,720,483, each of which are herein incorporated by reference.

Many CE products are commercially available. Preferred among these is the naturally occurring CE product known as Premarin® (Wyeth, Madison, N.J.). Another commercially available CE product prepared from synthetic estrogens is Cenestin® (Duramed Pharmaceuticals, Inc., Cincinnati, Ohio). The specific CE dose included in the core material may be any dosage required to achieve a specific therapeutic effect, and may vary depending on the specific treatment indicated, and on the specific CE included in the tablet.

In some embodiments, the first active agent comprises conjugated estrogens, and the second active agent comprises bazedoxifene or a salt thereof.

The materials, methods, and examples presented herein are intended to be illustrative, and are not intended to limit the scope of the invention.

### EXAMPLES

### Example 1

### Procedure for Preparation of Tablets Containing Conjugated Estrogens/Bazedoxifene Acetate; 0.45 mg/10 mg

A. Preparation of 10% w/w Stock Solution of Hydroxypropyl Methylcellulose, USP, 2910, 3 cps.
1.900 g of water, purified, USP, was placed in a vessel equipped with a low shear (Lightnin type) mixer.
2. 100 g of Hydroxypropyl Methylcellulose, USP, 2910, 3 cps., was added in small increments to the vortex created in the water by the moderate agitation of step 1.
3. Agitation was continued for am minimum of one hour, or until all the Hydroxypropyl Methylcellulose, USP, 2910, 3 cps., is dissolved.
4. If necessary, add water, purified USP to achieve the total theoretical weight.

B1. Preparation of Bazedoxifene Acetate Filler Coat Suspension:
1. 428.30 g of water, purified, USP was placed in a vessel equipped with a low shear (Lightnin type) and high shear (Silverson type) mixer.
2. With the Lightnin type mixer turned on, the water was heated to 60-70° C. (target 65° C.).
3. With the Lightnin type mixer turned on at slow speed, 110.64 g of sucrose, NF was added, and the mixture was reheated to 65° C. and held at that temperature for 10 minutes, until the sucrose dissolved.
4. With the Lightnin type mixer turned on at slow speed, 2.5523 g of sucrose palmitate was added, and mixing was continued at slow speed for 10 minutes. If necessary, mixing can be performed at high shear for 2 minutes with the high shear mixer to disperse and dissolve any undissolved sucrose palmitate.
5. The mixture was then cooled to 23-27° C. (target 25° C.). When the mixture was about 30° C., 4.0837 g of ascorbic acid, USP was slowly added, and mixing was continued using the Lightnin type mixer for 10 minutes.
6. When the mixture reached 23-27° C. (target 25° C.), mixing was ceased, and 46.054 g of bazedoxifene acetate was added. The low shear and high shear mixers were both turned on, and mixing was continued for approximately 15 minutes.
7. Both mixers were then turned off, the walls and shaft of the mixer were scraped, and both mixers were then turned on for an additional 15 minutes.
8. Both mixers were then turned off, and a sufficient amount of the 10% w/w stock solution of Hydroxypropyl Methylcellulose, USP, 2910, 3 cps. from Step B above was added to provide 408.37 g of Hydroxypropyl Methylcellulose, USP, 2910, 3 cps. The mixture was mixed at low speed for 10 minutes, adjusting the speed of the mixer to prevent generation of excessive foam.
9. The slurry from Step 8 above was brought to Theoretical Weight with water, purified, USP, and mixing was continued for an additional 10 minutes with the Lightnin type mixer at low speed.
10. Mixing of the suspension was continued with the Lightnin type mixer at low speed, and intermittently with the high shear mixer, as needed, while maintaining the temperature at 23-27° C. (target 25° C.). Mixing was continued with the low shear mixer until the level of the suspension fell below the agitator head.

B2. Alternate Procedure for Preparation of Bazedoxifene Acetate Filler Coat Suspension:
1. Water, USP, purified was placed in a suitable vessel equipped with a low shear (Lightnin type) and high shear (Silverson type or equivalent) mixers.
2. With the Lightnin type mixer turned on, the water was heated to 55-65° C. (target 60° C.).
3. The speed of the Lightnin mixer was adjusted to create a vortex without drawing air into the water. The sucrose palmitate was slowly added to the vortex and mixed for approximately 20 minutes to dissolve it. The high shear mixer was used for approximately 5 minutes to disperse and dissolve any undissolved particles of sucrose palmitate.
4. With the Lightnin type mixer turned on, Sucrose, NF was added to the vortex and reheated to 55-65° C. (target 60° C.), and then mixed for approximately 15 minutes to dissolve the sucrose NF. The temperature was then held at 60° C. for approximately 15 minutes, to ensure all the sucrose had dissolved.
5. With the Lightnin type mixer turned on, Hydroxypropyl Methylcellulose, USP, 2910, 3 cps was added to the vortex and reheated to 55-65° C. (target 60° C.). The high shear mixer could be used during and after the addition to aid the wetting of the Hydroxypropyl Methylcellulose, USP, 2910, 3 cps. The dispersion was mixed for approximately 15 minutes to disperse Hydroxypropyl Methylcellulose, USP, 2910, 3 cps, with the speed of the mixer being adjusted to obtain adequate mixing. The temperature was held at 60° C. for approximately 15 minutes to ensure all the Hydroxypropyl Methylcellulose, USP, 2910, 3 cps was well dispersed without forming any clumps.
6. With the Lightnin type mixer turned on, the solution from the preceding step was cooled to 23-27° C. (target 25° C.). Once the temperature was decreased below 30° C., the ascorbic acid, USP was slowly added, and mixing was continued using the Lightnin type mixer for approximately 10 minutes to dissolve ascorbic acid, USP.
7. When the temperature reached 23-27° C. (target 25° C.), the bazedoxifene acetate was added to the vortex. Once all the bazedoxifene acetate had been added, the high shear mixer was turned on and mixing was continued for approximately 10 minutes, with the power of the high shear mixer being adjusted not to generate excessive foaming.
8. Both the mixers were then turned off, and the tank walls and mixer shaft were scraped. Both mixers were then turned on at the same settings as were used in the previous step, and mixing continued for approximately 10 minutes to disperse the bazedoxifene acetate.
9. The suspension from the previous step was brought to theoretical Weight with water, USP, Purified, as necessary, and mixing was continued for an additional 10 minutes with the Lightnin type mixer at slow speed.
10. Mixing of the suspension with the Lightnin type mixer and intermittently with the high shear mixer (as needed) was continued during application, while maintaining temperature at 23-27° C. (target 25° C.). Mixing was performed continuously with the low shear mixer until the level of the suspension fell below the agitator head.

C. Preparation of Color Coat Suspension:
1. 675 g of water, purified, USP, was placed in a vessel equipped with a low shear (Lightnin type) and high shear (Silverson type) mixer.
2. With the Lightnin type mixer turned on, the water was heated to 60-70° C. (target 65° C.).
3. The speed of the Lightnin type mixer was adjusted to create a vortex without drawing air into the water. 300 g of sucrose, NF was added to the vortex, and the mixture was reheated to 60-70° C. (target 65° C.), mixed approximately 15 minutes to dissolve the sucrose, NF, and then held at that temperature for 15 minutes, making sure that all the sucrose was dissolved.
4. With the Lightnin type mixer turned on, 10 g of Hydroxypropyl Methylcellulose, USP, 2910, 3 cps. was added to the vortex, and the mixture was reheated to 60-70° C. (target 65° C.). The high shear mixer was used during and after the addition to aid the wetting of the Hydroxypropyl Methylcellulose, USP, 2910, 3 cps. Mixing was continued for 5 minutes, adjusting the speed of the mixer to obtain adequate mixing. The mixture is held at 65° C. for 5 minutes, making sure that all the Hydroxypropyl Methylcellulose, USP, 2910, 3 cps was well dispersed without forming clumps.
5. With the Lightnin type mixer turned on, the mixture was cooled to 23-27° C. (target 25° C.), bringing the mixture to weight with water, purified, USP as needed.
6. No more than 2 hours before the color suspension was to be applied to the tablets, 15 g of titanium dioxide, NF was added to the mixture, which was then mixed with the high shear mixer and low shear mixer for 10 minutes.
7. Both mixers were then turned off, the walls and shaft of the mixer were scraped, and both mixers were then turned on for an additional 5 minutes. Mixing was continued with the low shear mixer during color application.

D. Preparation of Clear Coat Suspension:
1. 950 g of water, purified, USP, was placed in a stainless steel container equipped with a low shear (Lightnin type) mixer.
2. With the propeller in the center and as close to the bottom of the vessel as possible, the mixture was stirred to form a vortex without drawing air into the liquid.
3. 50.0 g of Opadry clear, YS-2-19114 A, was added slowly, avoiding powder floatation on the liquid surface. The stirrer speed was increased to maintain the vortex as required.

E. Tablet Coating Procedure:

1. Premarin® 0.45 mg No Talc Triturate Filled tablets were loaded into a perforated coating pan.

2. Sufficient Bazedoxifene Acetate Filler Coat Suspension from Step C above was added to achieve a total weight of 50 mg (±2 mg) above the inert tablet filled weight (approximately 244.9 mg of Bazedoxifene Acetate Filler Coat Suspension per tablet). The tablets were coated with the suspension using a continuous coating technique, wherein the suspension is sprayed on to the tablets in the rotating coating pan, with concomitant drying by hot air, until the target weight gain is achieved.

3. Approximately 15 mg of the Color Coat was applied to the tablets (approximately 46.15 mg of color coat suspension per tablet).

4. Approximately 3 mg of the Clear Coat was applied to the tablets (approximately 60 mg of clear coat suspension per tablet).

The composition of the tablets is shown in the Table below.

| Ingredient | % WT/WT | mg/tablet | |
|---|---|---|---|
| Tablet Core | | | |
| Premarin ® 0.45 mg, No Talc Triturate, | 77.56 | 235.0 | mg |
| Filled Tablets | | | |
| Bazedoxifene Coat | | | |
| Sucrose, NF | 8.94 | 27.10 | mg |
| Hydroxypropyl Methylcellulose, | 3.30 | 10.00 | mg |
| USP, 2910, 3 cps (Pharmacoat 603) | | | |
| Water, USP Purified^{a} | | 194.9 | mg |
| Sucrose Palmitate | 0.21 | 0.625 | mg |
| Ascorbic Acid, USP | 0.33 | 1.000 | mg |
| Bazedoxifene Acetate (88.68% | 3.72 | 11.28 | mg |
| bazedoxifene free base)^{c} | | | |
| Color Coat^{b} | | | |
| Sucrose, NF | 4.57 | 13.85 | mg |
| Hydroxypropyl Methylcellulose, | 0.15 | 0.46 | mg |
| USP, 2910, 3 cps (Pharmacoat 603) | | | |
| Titanium Dioxide, USP | 0.23 | 0.69 | mg |
| Water, USP Purified^{a} | | 31.15 | mg |
| Clear Coat^{b} | | | |
| Opadry Clear YS-2-19114A | 0.99 | 3.00 | mg |
| Water, USP Purified^{a} | | 57.00 | mg |
| TOTAL | 100.00 | 303 | mg |

^{a}Removed during processing.
^{b}The quantities of the ingredients per tablet represent theoretical amounts of coating solids applied. For elegance, the amount of water and coating ingredients used may vary. These should not exceed +/−10% of the theoretical values.
^{c}The potency of bazedoxifene acetate may vary, and the amount in the formula must be adjusted accordingly with a corresponding adjustment in the amount of Sucrose.

### Example 2

### Procedure for Preparation of Tablets Containing Conjugated Estrogens/Bazedoxifene Acetate; 0.45 mg/20 mg

The procedure is essentially as described for Example 1, except that:

a) the Color Coat Suspension contained:
125.00 g of Opadry White YS-1-18202 A;
875.00 g of water, USP, purified; and

b) the Clear Coat Suspension contained:
50.00 g of Opadry Clear YS-2-19114 A;
950.00 g of water, USP, purified; and

c) the following tablet coating procedure was used:

1. Premarin® 0.45 mg No Talc Triturate Filled tablets were loaded into a perforated coating pan.

2. Sufficient Bazedoxifene Acetate Filler Coat Suspension was added to achieve a total weight of 100 mg (±2 mg) above the inert tablet filled weight (approximately 489.8 mg of Bazedoxifene Acetate Filler Coat Suspension per tablet). The tablets were coated with the suspension using the continuous sugar coating technique as described in Example 1.

3. Approximately 17 mg of the Color Coat was applied to the tablets (approximately 136 mg of color coat suspension per tablet).

4. Approximately 4 mg of the Clear Coat was applied to the tablets (approximately 80 mg of clear coat suspension per tablet).

The composition of the tablets is shown in the Table below.

| Ingredient | % WT/WT | mg/tablet |
|---|---|---|
| Tablet Core | | |
| Premarin ® 0.45 mg, No Talc Triturate, | 66.01 | 235.0 mg |
| Filled Tablets | | |
| Bazedoxifene Coat | | |
| Sucrose, NF | 15.22 | 54.19 mg |
| Hydroxypropyl Methylcellulose, | 5.62 | 20.00 mg |
| USP, 2910, 3 cps (Pharmacoat 603) | | |
| Water, USP Purified^{a} | | 389.8 mg |
| Sucrose Palmitate | 0.35 | 1.250 mg |
| Ascorbic Acid, USP | 0.56 | 2.000 mg |
| Bazedoxifene Acetate (88.68% | 6.34 | 22.56 mg |
| bazedoxifene free base)^{c} | | |
| Color Coat^{b} | | |
| Opadry White YS-1-18202 A | 4.78 | 17.00 mg |
| Water, USP Purified^{a} | | 119.0 mg |
| Clear Coat^{b} | | |
| Opadry Clear YS-2-19114A | 1.12 | 4.00 mg |
| Water, USP Purified^{a} | | 76.00 mg |
| TOTAL | 100.00 | 356 mg |

^{a}Removed during processing.
^{b}The quantities of the ingredients per tablet represent theoretical amounts of coating solids applied. For elegance, the amount of water and coating ingredients used may vary. These should not exceed +/−10% of the theoretical values.
^{c}The potency of bazedoxifene acetate may vary, and the amount in the formula must be adjusted accordingly with a corresponding adjustment in the amount of Sucrose.

### Example 3

### Procedure for Preparation of Tablets Containing Conjugated Estrogens/Bazedoxifene Acetate; 0.45 mg/40 mg

The procedure is essentially as described for Example 1, except that:

a) the Color Coat Suspension contained:
125.00 g of Opadry White YS-1-18202 A;
875.00 g of water, USP, purified; and

b) the Clear Coat Suspension contained:
50.00 g of Opadry Clear YS-2-19114 A;
950.00 g of water, USP, purified; and

c) the following tablet coating procedure was used:

1. Premarin® 0.45 mg No Talc Triturate Filled tablets were loaded into a perforated coating pan.

2. Sufficient Bazedoxifene Acetate Filler Coat Suspension was added to achieve a total weight of 200 mg (±2 mg) above the inert tablet filled weight (approximately 979.6 mg of Bazedoxifene Acetate Filler Coat Suspension per tablet). The tablets were coated with the suspension using the continuous sugar coating technique as described in Example 1.

3. Approximately 22 mg of the Color Coat was applied to the tablets (approximately 176 mg of color coat suspension per tablet).

4. Approximately 5 mg of the Clear Coat was applied to the tablets (approximately 100 mg of clear coat suspension per tablet).

The composition of the tablets is shown in the Table below.

| Ingredient | % WT/WT | mg/tablet |
|---|---|---|
| Tablet Core | | |
| Premarin ® 0.45 mg, No Talc Triturate, | 50.87 | 235.0 mg |
| Filled Tablets | | |
| Bazedoxifene Coat | | |
| Sucrose, NF | 23.46 | 108.38 mg |
| Hydroxypropyl Methylcellulose, | 8.66 | 40.00 mg |
| USP, 2910, 3 cps (Pharmacoat 603) | | |
| Water, USP Purified^{a} | | 779.6 mg |
| Sucrose Palmitate | 0.54 | 2.50 mg |
| Ascorbic Acid, USP | 0.87 | 4.000 mg |
| Bazedoxifene Acetate (88.68% | 9.77 | 45.11 mg |
| bazedoxifene free base)^{c} | | |
| Color Coat^{b} | | |
| Opadry White YS-1-18202 A | 4.76 | 22.00 mg |
| Water, USP Purified^{a} | | 154.0 mg |
| Clear Coat^{b} | | |
| Opadry Clear YS-2-19114A | 1.08 | 5.00 mg |
| Water, USP Purified^{a} | | 95.00 mg |
| TOTAL | 100.00 | 462 mg |

^{a}Removed during processing.
^{b}The quantities of the ingredients per tablet represent theoretical amounts of coating solids applied. For elegance, the amount of water and coating ingredients used may vary. These should not exceed +/−10% of the theoretical values.
^{c}The potency of bazedoxifene acetate may vary, and the amount in the formula must be adjusted accordingly with a corresponding adjustment in the amount of Sucrose.

### Example 4

### Procedure for Preparation of Tablets Containing Conjugated Estrogens/Bazedoxifene Acetate; 0.625 mg/10 mg

The procedure is essentially as described for Example 1, except that tablets to which the coatings were applied were Premarin® 0.625 mg, No Talc Triturate, Filled Tablets.

The composition of the tablets is shown in the Table below.

| Ingredient | % WT/WT | mg/tablet |
|---|---|---|
| Tablet Core | | |
| Premarin ® 0.625 mg, No Talc | 77.56 | 235.0 mg |
| Triturate, | | |
| Filled Tablets | | |
| Bazedoxifene Coat | | |
| Sucrose, NF | 8.94 | 27.10 mg |
| Hydroxypropyl Methylcellulose, | 3.30 | 10.00 mg |
| USP, 2910, 3 cps (Pharmacoat 603) | | |
| Water, USP Purified^{a} | | 194.9 mg |
| Sucrose Palmitate | 0.21 | 0.625 mg |
| Ascorbic Acid, USP | 0.33 | 1.000 mg |
| Bazedoxifene Acetate (88.68% | 3.72 | 11.28 mg |
| bazedoxifene free base)^{c} | | |
| Color Coat^{b} | | |
| Sucrose, NF | 4.57 | 13.85 mg |
| Hydroxypropyl Methylcellulose, | 0.15 | 0.46 mg |
| USP, 2910, 3 cps (Pharmacoat 603) | | |
| Titanium Dioxide, USP | 0.23 | 0.69 mg |
| Water, USP Purified^{a} | | 31.15 mg |
| Clear Coat^{b} | | |
| Opadry Clear YS-2-19114A | 0.99 | 3.00 mg |
| Water, USP Purified^{a} | | 57.00 mg |
| TOTAL | 100.00 | 303 mg |

^{a}Removed during processing.
^{b}The quantities of the ingredients per tablet represent theoretical amounts of coating solids applied. For elegance, the amount of water and coating ingredients used may vary. These should not exceed +/−10% of the theoretical values.
^{c}The potency of bazedoxifene acetate may vary, and the amount in the formula must be adjusted accordingly with a corresponding adjustment in the amount of Sucrose.

### Example 5

### Procedure for Preparation of Tablets Containing Conjugated Estrogens/Bazedoxifene Acetate; 0.625 mg/20 mg

The procedure is essentially as described for Example 2, except that tablets to which the coatings were applied were Premarin® 0.625 mg, No Talc Triturate, Filled Tablets.

The composition of the tablets is shown in the Table below.

| Ingredient | % WT/WT | mg/tablet |
|---|---|---|
| Tablet Core | | |
| Premarin ® 0.625 mg, No Talc | 66.01 | 235.0 mg |
| Triturate, | | |
| Filled Tablets | | |
| Bazedoxifene Coat | | |
| Sucrose, NF | 15.22 | 54.19 mg |
| Hydroxypropyl Methylcellulose, | 5.62 | 20.00 mg |
| USP, 2910, 3 cps (Pharmacoat 603) | | |
| Water, USP Purified^{a} | | 389.8 mg |
| Sucrose Palmitate | 0.35 | 1.250 mg |
| Ascorbic Acid, USP | 0.56 | 2.000 mg |
| Bazedoxifene Acetate (88.68% | 6.34 | 22.56 mg |
| bazedoxifene free base)^{c} | | |
| Color Coat^{b} | | |
| Opadry White YS-1-18202 A | 4.78 | 17.00 mg |
| Water, USP Purified^{a} | | 119.0 mg |
| Clear Coat^{b} | | |
| Opadry Clear YS-2-19114A | 1.12 | 4.00 mg |
| Water, USP Purified^{a} | | 76.00 mg |
| TOTAL | 100.00 | 356 mg |

^{a}Removed during processing.
^{b}The quantities of the ingredients per tablet represent theoretical amounts of coating solids applied. For elegance, the amount of water and coating ingredients used may vary. These should not exceed +/−10% of the theoretical values.
^{c}The potency of bazedoxifene acetate may vary, and the amount in the formula must be adjusted accordingly with a corresponding adjustment in the amount of Sucrose.

### Example 6

### Procedure for Preparation of Tablets Containing Conjugated Estrogens/Bazedoxifene Acetate; 0.625 mg/40 mg

The procedure is essentially as described for Example 3, except that tablets to which the coatings were applied were Premarin® 0.625 mg, No Talc Triturate, Filled Tablets.

The composition of the tablets is shown in the Table below.

| Ingredient | % WT/WT | mg/tablet |
|---|---|---|
| Tablet Core | | |
| Premarin ® 0.625 mg, No Talc | 50.87 | 235.0 mg |
| Triturate, | | |
| Filled Tablets | | |
| Bazedoxifene Coat | | |
| Sucrose, NF | 23.46 | 108.38 mg |
| Hydroxypropyl Methylcellulose, | 8.66 | 40.00 mg |
| USP, 2910, 3 cps (Pharmacoat 603) | | |
| Water, USP Purified^{a} | | 779.6 mg |
| Sucrose Palmitate | 0.54 | 2.50 mg |
| Ascorbic Acid, USP | 0.87 | 4.000 mg |
| Bazedoxifene Acetate (88.68% | 9.77 | 45.11 mg |
| bazedoxifene free base)^{c} | | |
| Color Coat^{b} | | |
| Opadry White YS-1-18202 A | 4.76 | 22.00 mg |
| Water, USP Purified^{a} | | 154.0 mg |
| Clear Coat^{b} | | |
| Opadry Clear YS-2-19114A | 1.08 | 5.00 mg |
| Water, USP Purified^{a} | | 95.00 mg |
| TOTAL | 100.00 | 462 mg |

^{a}Removed during processing.
^{b}The quantities of the ingredients per tablet represent theoretical amounts of coating solids applied. For elegance, the amount of water and coating ingredients used may vary. These should not exceed +/−10% of the theoretical values.
^{c}The potency of bazedoxifene acetate may vary, and the amount in the formula must be adjusted accordingly with a corresponding adjustment in the amount of Sucrose.

It is intended that each of the patents, applications, and printed publications including books mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A pharmaceutical composition comprising a core and at least one coating; wherein the core comprises conjugated estrogens; and the coating comprises bazedoxifene, or a pharmaceutically acceptable salt thereof.

2. The pharmaceutical composition of claim 1, wherein the pharmaceutical composition is a tablet.

3. The pharmaceutical composition of claim 2, wherein the conjugated estrogens comprise Premarin®.

4. The pharmaceutical composition of claim 2, wherein the conjugated estrogens is present in an amount of from about 0.10 to about 1.0 mg.

5. The pharmaceutical composition of claim 2 wherein the conjugated estrogens is present in an amount of from about 0.3 to about 0.8 mg.

6. The pharmaceutical composition of claim 2 wherein the conjugated estrogens is present in an amount of from about 0.4 to about 0.5 mg.

7. The pharmaceutical composition of claim 2 wherein the conjugated estrogens is present in an amount of from about 0.5 to about 0.7 mg.

8. The pharmaceutical composition of claim 2 wherein the bazedoxifene is present in an amount of from about 1 to about 50 mg, based on the weight of the bazedoxifene free base.

9. The pharmaceutical composition of claim 2 wherein the bazedoxifene is present in an amount of from about 5 to about 25 mg, based on the weight of the bazedoxifene free base.

10. The pharmaceutical composition of claim 2 wherein the bazedoxifene is present in an amount of from about 5 to about 15 mg, based on the weight of the bazedoxifene free base.

11. The pharmaceutical composition of claim 2 wherein the bazedoxifene is present in an amount of from about 15 to about 25 mg, based on the weight of the bazedoxifene free base.

12. The pharmaceutical composition of claim 2 wherein the bazedoxifene is present in an amount of from about 35 to about 45 mg, based on the weight of the bazedoxifene free base.

13. The pharmaceutical composition of claim 2 wherein the conjugated estrogens is present in an amount of from about 0.10 to about 1.0 mg; and the bazedoxifene is present in an amount of from about 5 to about 50 mg, based on the weight of the bazedoxifene free base.

14. The pharmaceutical composition of claim 10 wherein the conjugated estrogens is present in an amount of from about 0.4 to about 0.5 mg.

15. The pharmaceutical composition of claim 10 wherein the conjugated estrogens is present in an amount of from about 0.5 to about 0.7 mg.

16. The pharmaceutical composition of claim 11 wherein the conjugated estrogens is present in an amount of from about 0.4 to about 0.5 mg.

17. The pharmaceutical composition of claim 11 wherein the conjugated estrogens is present in an amount of from about 0.5 to about 0.7 mg.

18. The pharmaceutical composition of claim 12 wherein the conjugated estrogens is present in an amount of from about 0.4 to about 0.5 mg.

19. The pharmaceutical composition of claim 12 wherein the conjugated estrogens is present in an amount of from about 0.5 to about 0.7 mg.

20. The pharmaceutical composition of claim 12 wherein the conjugated estrogens is Premarin®, and the bazedoxifene is bazedoxifene acetate.

21. A pharmaceutical composition of claim 2 wherein the coating comprises:
a) a filler component comprising from about 5% to about 30% by weight of the pharmaceutical formulation;
b) a binder component comprising from about 1% to about 10% by weight of the pharmaceutical formulation;
c) a wetting agent component comprising from about 0.01% to about 2% by weight of the pharmaceutical formulation;
d) an optional antioxidant component comprising from 0% to about 2% by weight of the pharmaceutical formulation;
e) bazedoxifene acetate, comprising from about 0.1% to about 20% by weight of the pharmaceutical formulation; and
f) an optional chelating component comprising from 0% to about 0.1% by weight of the pharmaceutical formulation.

22. The pharmaceutical composition of claim 21 wherein the core comprises from about 45% to about 80% by weight of the pharmaceutical formulation.

23. The pharmaceutical composition of claim 21 wherein the core comprises conjugated estrogens in an amount of from about 0.10 to about 1.0 mg.

24. The pharmaceutical composition of claim 21 wherein the composition contains from about 1 mg to about 50 mg of bazedoxifene, based on the weight of the bazedoxifene free base.

25. The pharmaceutical composition of claim 21 wherein the composition contains from about 5 mg to about 25 mg of bazedoxifene, based on the weight of the bazedoxifene free base.

26. The pharmaceutical composition of claim 21 wherein the composition contains from about 0.3 mg to about 0.8 mg of conjugated estrogens.

27. The pharmaceutical composition of claim 21 wherein the composition contains from about 0.4 mg to about 0.5 mg of conjugated estrogens.

28. The pharmaceutical composition of claim 21 wherein the composition contains from about 0.5 mg to about 0.7 mg of conjugated estrogens.

29. The pharmaceutical composition of claim 21 wherein the conjugated estrogens is present in an amount of from about 0.10 to about 1.0 mg; and the bazedoxifene is present in an amount of from about 5 to about 25 mg, based on the weight of the bazedoxifene free base.

30. The pharmaceutical composition of claim 2 wherein the coating comprises:
a) a filler component comprising from about 6% to about 12% by weight of the pharmaceutical formulation;
b) a binder component comprising from about 1% to about 6% by weight of the pharmaceutical formulation;
c) a wetting agent component comprising from about 0.1% to about 3% by weight of the pharmaceutical formulation;
d) an optional antioxidant component comprising from 0% to about 0.5% by weight of the pharmaceutical formulation;
e) bazedoxifene acetate, comprising from about 2% to about 6% by weight of the pharmaceutical formulation; and
f) an optional chelating component comprising from 0% to about 0.1% by weight of the pharmaceutical formulation.

31. The pharmaceutical composition of claim 30 wherein the composition contains from about 0.3 mg to about 0.8 mg of conjugated estrogens.

32. The pharmaceutical composition of claim 30 wherein the composition contains from about 0.4 mg to about 0.5 mg of conjugated estrogens.

33. The pharmaceutical composition of claim 21 wherein the coating comprises:
a) a filler component comprising from about 12% to about 18% by weight of the pharmaceutical formulation;
b) a binder component comprising from about 4% to about 8% by weight of the pharmaceutical formulation;
c) a wetting agent component comprising from about 0.2% to about 0.5% by weight of the pharmaceutical formulation;
d) an optional antioxidant component comprising, when present, from about 0.3% to about 0.8% by weight of the pharmaceutical formulation;
e) bazedoxifene acetate, comprising from about 4% to about 9% by weight of the pharmaceutical formulation; and
f) an optional chelating component comprising from 0% to about 0.1% by weight of the pharmaceutical formulation.

34. The pharmaceutical composition of claim 33 wherein the composition contains from about 0.3 mg to about 0.8 mg of conjugated estrogens.

35. The pharmaceutical composition of claim 33 wherein the composition contains from about 0.4 mg to about 0.5 mg of conjugated estrogens.

36. The pharmaceutical composition of claim 2 wherein the coating comprises:
a) a filler component comprising from about 20% to about 30% by weight of the pharmaceutical formulation;
b) a binder component comprising from about 6% to about 10% by weight of the pharmaceutical formulation;
c) a wetting agent component comprising from about 0.4% to about 0.8% by weight of the pharmaceutical formulation;
d) an optional antioxidant component comprising from 0% to about 1.2% by weight of the pharmaceutical formulation;
e) bazedoxifene acetate, comprising from about 7% to about 14% by weight of the pharmaceutical formulation; and
f) an optional chelating component comprising from 0% to about 0.1% by weight of the pharmaceutical formulation.

37. The pharmaceutical composition of claim 36 wherein the composition contains from about 0.3 mg to about 0.8 mg of conjugated estrogens.

38. The pharmaceutical composition of claim 36 wherein the composition contains from about 0.4 mg to about 0.5 mg of conjugated estrogens.

39. The pharmaceutical composition of claim 21 wherein:
the filler component of the coating comprises one or more of sucrose, mannitol, lactose, powdered cellulose, microcrystalline cellulose, malodextrin, sorbitol, starch, xylitol, carboxymethyl cellulose, carboxyethyl cellulose, hydroxyethyl celluloses, microcrystalline celluloses, starches, anhydrous dicalcium phosphate, sodium starch glycolates, and metal aluminosilicates;
the binder component of the coating comprises one or more of hydroxypropylmethylcellulose, carboxymethyl cellulose, carboxyethyl cellulose, hydroxyethyl celluloses, microcrystalline celluloses, starches and polyvinyl pyrrolidine;
the wetting agent component of the coating comprises one or more of sucrose palmitate, Poloxamer 188, metal alkyl sulfates, sodium lauryl sulfate, polyoxyethylene sorbitan fatty acid esters, polyethylene glycols, polyoxyethylene castor oil derivatives, docusate sodium, quaternary ammonium amine compounds, sugar esters of fatty acids and glycerides of fatty acids; and
the optional antioxidant component of the coating, when present, comprises one or more of ascorbic acid or a salt thereof, sodium ascorbate, citric acid, ascorbyl palmitate, nicotinamide ascorbate, propyl gallate, alpha tocopherol, beta tocopherol, gamma tocopherol, and BHA/BHT; and
the optional chelating component of the coating, when present, comprises EDTA.

40. The pharmaceutical composition of claim 21 wherein:
the filler component of the coating comprises sucrose;
the binder component of the coating comprises hydroxypropylmethylcellulose;
the wetting agent component of the coating comprises sucrose palmitate;
the optional antioxidant component of the coating, when present, comprises ascorbic acid, or a salt thereof; and
the optional chelating component of the coating, when present, comprises EDTA.

41. The pharmaceutical composition of claim 21 further comprising a color coating.

42. The pharmaceutical composition of claim 41 wherein the color coating comprises:
a) an optional filler component comprising from about 0.01% to about 8% by weight of the pharmaceutical formulation;
b) an optional binder component comprising from about 0.01% to about 2% by weight of the pharmaceutical formulation;
c) a coloring agent component comprising from about 0.01% to about 6% by weight of the pharmaceutical formulation;
d) an optional antioxidant component comprising from 0% to about 2% by weight of the pharmaceutical formulation; and
e) an optional chelating component comprising from 0% to about 0.1% by weight of the pharmaceutical formulation.

43. The pharmaceutical composition of claim 42 wherein:
the optional filler component comprises sucrose;
the optional binder component comprises hydroxypropylmethylcellulose; and
the coloring agent component comprises titanium dioxide;
the optional antioxidant component comprises ascorbic acid or a salt thereof; and
the optional chelating component comprises EDTA.

44. The pharmaceutical composition of claim 21 further comprising a clear coating.

45. The pharmaceutical composition of claim 42 further comprising a clear coating.

46. The pharmaceutical composition of claim 44 wherein the clear coating comprises from about 0.01% to about 2% by weight of the pharmaceutical formulation.

47. The pharmaceutical composition of claim 35 wherein the clear coating comprises from about 0.01% to about 2% by weight of the pharmaceutical formulation.

48. A pharmaceutical composition comprising:
a core comprising conjugated estrogens in an amount of from about 0.10 mg to about 1.0 mg;
a first coating;
a color coating; and
a clear coating;
wherein:
the core comprises from about 45% to about 80% by weight of the pharmaceutical formulation;
the first coating comprises:
a) a filler component comprising from about 5% to about 30% by weight of the pharmaceutical formulation;
b) a binder component comprising from about 1% to about 10% by weight of the pharmaceutical formulation;
c) a wetting agent component comprising from about 0.01% to about 2% by weight of the pharmaceutical formulation;
d) an optional antioxidant component comprising from 0% to about 2% by weight of the pharmaceutical formulation;
e) bazedoxifene acetate, comprising from about 0.1% to about 20% by weight of the pharmaceutical formulation; and
f) an optional chelating component comprising from 0% to about 0.1% by weight of the pharmaceutical formulation;
the color coating comprises:
g) an optional filler component comprising from about 0.01% to about 8% by weight of the pharmaceutical formulation;
h) an optional binder component comprising from about 0.01% to about 2% by weight of the pharmaceutical formulation;
i) a coloring agent component comprising from about 0.01% to about 6% by weight of the pharmaceutical formulation;
j) an optional antioxidant component comprising from 0% to about 2% by weight of the pharmaceutical formulation;
k) an optional chelating component comprising from 0% to about 0.1% by weight of the pharmaceutical formulation;
the clear coating comprising from about 0.01% to about 2% by weight of the pharmaceutical formulation; said clear coating comprising an optional antioxidant component comprising from 0% to about 2% by weight of the pharmaceutical formulation; and an optional chelating component comprising from 0% to about 0.1% by weight of the pharmaceutical formulation.

49. The pharmaceutical composition of claim 48 wherein the composition contains from about 1 mg to about 50 mg of bazedoxifene, based on the weight of the bazedoxifene free base.

50. The pharmaceutical composition of claim 49 wherein:
the filler component of the first coating comprises sucrose;
the binder component of the first coating comprises hydroxypropylmethylcellulose;
the wetting agent component of the first coating comprises sucrose palmitate;
the optional antioxidant component of the first coating, when present, comprises ascorbic acid or a salt thereof;
the optional chelating component of the first coating, when present, comprises EDTA;
the optional filler component of the color coating, when present, comprises sucrose;
the optional binder component of the color coating, when present, comprises hydroxypropylmethylcellulose;
the optional antioxidant component of the color coating, when present, comprises ascorbic acid or a salt thereof;
the optional chelating component of the color coating, when present, comprises EDTA; and
the coloring agent component of the color coating comprises titanium dioxide.

51. A process for preparing a pharmaceutical composition, the composition comprising:
a core comprising conjugated estrogens; and
a first coating comprising bazedoxifene or a pharmaceutically acceptable salt thereof;
the process comprising:
i) providing a core comprising conjugated estrogens; and
ii) coating the core with a coating composition comprising bazedoxifene or a pharmaceutically acceptable salt thereof to form a coated core.

52. A process of claim 51 further comprising the step of:
iii) coating the coated core with a color coating composition to form a color coated composition.

53. A process of claim 52 further comprising the step of:
iv) coating the color coated composition with a clear coating composition to form a clear coat thereon.

54. A process of claim 51 wherein the coating composition of step ii comprises:
a) a filler component;
b) a binder component;
c) a wetting agent component;
d) an optional antioxidant component;
e) bazedoxifene acetate; and
f) an optional chelating component.

55. A process of claim 54 wherein:
the filler component comprises one or more of sucrose, mannitol, lactose, powdered cellulose, microcrystalline cellulose, malodextrin, sorbitol, starch, xylitol, carboxymethyl cellulose, carboxyethyl cellulose, hydroxyethyl celluloses, microcrystalline celluloses, starches, anhydrous dicalcium phosphate, sodium starch glycolates, and metal aluminosilicates;
the binder component comprises one or more of hydroxypropylmethylcellulose, carboxymethyl cellulose, carboxyethyl cellulose, hydroxyethyl celluloses, microcrystalline celluloses, starches and polyvinyl pyrrolidine;
the wetting agent component comprises one or more of sucrose palmitate, Poloxamer 188, metal alkyl sulfates, sodium lauryl sulfate, polyoxyethylene sorbitan fatty acid esters, polyethylene glycols, polyoxyethylene castor oil derivatives, docusate sodium, quaternary ammonium amine compounds, sugar esters of fatty acids and glycerides of fatty acids; and
the optional antioxidant component, when present, comprises one or more of ascorbic acid, sodium ascorbate, citric acid, ascorbyl palmitate, nicotinamide ascorbate, propyl gallate, alpha tocopherol, beta tocopherol, gamma tocopherol and BHA/BHT; and
the optional chelating component, when present, comprises EDTA.

56. A process of claim 54 wherein:
the filler component of the coating comprises sucrose;
the binder component of the coating comprises hydroxypropylmethylcellulose;
the wetting agent component of the coating comprises sucrose palmitate;
the optional antioxidant component of the coating, when present, comprises ascorbic acid, or a salt thereof; and
the optional chelating component of the coating, when present, comprises EDTA.

57. A process of claim 56 wherein the coating composition of step ii is applied by a continuous sugar coating technique.

58. A process of claim 52 wherein the color coating composition comprises:
an optional filler component;
an optional binder component;
a coloring agent component;
an optional antioxidant component; and
an optional chelating component.

59. A process of claim 58 wherein:
the optional filler component of the color coating composition comprises sucrose;
the optional binder component of the color coating composition comprises hydroxypropylmethylcellulose;
the coloring agent component of the color coating composition comprises titanium dioxide;
the optional antioxidant component of the color coating comprises ascorbic acid or a salt thereof; and
the optional chelating component of the color coating comprises EDTA.

60. A process of claim 56 wherein:
a) the filler component of the coating composition of step ii comprises from about 5% to about 30% by weight of the pharmaceutical formulation;
b) the binder component of the coating composition of step ii comprises from about 1% to about 10% by weight of the pharmaceutical formulation;
c) the wetting agent component of the coating composition of step ii comprises from about 0.01% to about 2% by weight of the pharmaceutical formulation;
d) the optional antioxidant component of the coating composition of step ii comprises from 0% to about 2% by weight of the pharmaceutical formulation;
e) the bazedoxifene acetate comprises from about 0.1% to about 20% by weight of the pharmaceutical formulation; and
f) the optional chelating component of the coating composition comprises from 0% to about 0.1% by weight of the pharmaceutical formulation.

61. A process of claim 60 wherein the conjugated estrogens is present in an amount of from about 0.10 to about 1.0 mg.

62. A process of claim 60 wherein the bazedoxifene is present in an amount of from about 1 to about 50 mg, based on the weight of the bazedoxifene free base.

63. A process of claim 60 wherein the conjugated estrogens is present in an amount of from about 0.10 to about 1.0 mg; and the bazedoxifene is present in an amount of from about 5 to about 50 mg, based on the weight of the bazedoxifene free base.

64. A pharmaceutical composition of any of claims 13, 21, and 48 wherein the conjugated estrogens comprise Premarin®.

65. A process of any of claims 51, 54, 56, 57 and 60 wherein the conjugated estrogens comprise Premarin®.

66. A product of the process of any of claims 51 and 57.

67. A process for preparing a pharmaceutical composition, the composition comprising:
a core comprising a therapeutic agent; and
a coating optionally comprising a second therapeutic agent and at least one sugar;
the process comprising:
i) providing a core comprising the first active agent; and
ii) coating the core with a coating composition comprising:
a) a filler component that comprises at least one sugar;
b) a binder component;
c) a wetting agent component;
d) an optional antioxidant component;
e) optionally, a second therapeutic agent; and
f) an optional chelating component;
wherein the coating composition of step ii is applied by a continuous sugar coating technique.

68. A process of claim 67 wherein:
the filler component comprises one or more of sucrose, mannitol, lactose, powdered cellulose, microcrystalline cellulose, malodextrin, sorbitol, starch, xylitol, carboxymethyl cellulose, carboxyethyl cellulose, hydroxyethyl celluloses, microcrystalline celluloses, starches, anhydrous dicalcium phosphate, sodium starch glycolates, and metal aluminosilicates;
the binder component comprises one or more of hydroxypropylmethylcellulose, carboxymethyl cellulose, carboxyethyl cellulose, hydroxyethyl celluloses, microcrystalline celluloses, starches and polyvinyl pyrrolidine;
the wetting agent component comprises one or more of sucrose palmitate, Poloxamer 188, metal alkyl sulfates, sodium lauryl sulfate, polyoxyethylene sorbitan fatty acid esters, polyethylene glycols, polyoxyethylene castor oil derivatives, docusate sodium, quaternary ammonium amine compounds, sugar esters of fatty acids and glycerides of fatty acids; and
the optional antioxidant component, when present, comprises one or more of ascorbic acid, sodium ascorbate, citric acid and ascorbyl palmitate, nicotinamide ascorbate, propyl gallate, alpha tocopherol, beta tocopherol, gamma tocopherol, and BHA/BHT; and
the optional chelating component, when present, comprises EDTA.

69. A process of claim 67 wherein:
the filler component of the coating comprises sucrose;
the binder component of the coating comprises hydroxypropylmethylcellulose;
the wetting agent component of the coating comprises sucrose palmitate;
the optional antioxidant component of the coating, when present, comprises ascorbic acid, or a salt thereof; and
the optional chelating component of the coating, when present, comprises EDTA.

70. A process of claim 67 further comprising the step of:
iii) coating the coated core with a color coating composition to form a color coated composition.

71. A process of claim 70 further comprising the step of:
iv) coating the color coated composition with a clear coating composition to form a clear coat thereon.

72. A process of claim 67 wherein the first therapeutic agent comprises conjugated estrogens, and the second therapeutic agent comprises bazedoxifene or a salt thereof.
